(19) United States
(12) Patent Application Publication
(10) Pub. No.: US 2024/0299430 A1
(43) Pub. Date: Sep. 12, 2024

(54) METHODS AND COMPOSITIONS FOR TREATING LONG COVID-19

(71) Applicant: Intrinsic Medicine, Inc., Seattle, WA (US)

(72) Inventors: Jason Ferrone, San Diego, CA (US); Alexander Martinez, Des Moines, WA (US)

(21) Appl. No.: 18/379,685

(22) Filed: Oct. 13, 2023

Related U.S. Application Data
(63) Continuation of application No. PCT/US22/24516, filed on Apr. 13, 2022.
(60) Provisional application No. 63/175,160, filed on Apr. 15, 2021.

(51) Int. Cl. A61K 31/702

(52) U.S. Cl. CPC A61K 31/702

(57) ABSTRACT

The invention provides methods of treating long COVID in a patient in need thereof, comprising administering to said patient a composition comprising a therapeutically effective amount of at least one human milk oligosaccharides, wherein such treatment reduces or improves at least one sign or symptom of long COVID.

### RELATED APPLICATIONS

This application is a continuation of International Application No. PCT/US22/24516, which designated the United States and was filed on Apr. 13, 2022, published in English, which claims the benefit of U.S. Provisional Application No. 63/175,160 filed Apr. 15, 2021. The entire contents of the above-referenced applications are incorporated by reference herein.

### BACKGROUND OF THE INVENTION

A substantial portion of patients infected with SARS-CoV2 (COVID-19) continue to suffer sequelae or post-COVID conditions that persist more than a few weeks after the early or acute phase of infection. These conditions or sequelae are referred to as “Long COVID.” Long COVID can appear weeks after infection and can persist for three or more weeks (Lopez-Leon et al. (2021). More than 50 Long-term effects of COVID-19: a systematic review and meta-analysis. medRxiv [Preprint]. 2021 Jan. 30:2021.01.27.21250617. doi: 10.1101/2021.01.27.21250617. PMID: 33532785; PMCID: PMC7852236.)

Seemingly disparate symptoms have been reported including fatigue, muscle weakness, brain fog, memory loss, anosmia, heart palpitations, fever, cough, hair loss, and shortness of breath (cdc.gov/coronavirus/2019-ncov/long-term-effects.html). It has been estimated that about 80% of patients that were infected with SARS-CoV-2 suffer at least one long-term symptom (Lopez-Leon et al. (2021)). Nalbandian et al. divides post-acute symptoms into two categories: subacute or ongoing symptoms present from 4-12 weeks after acute infection and chronic or post-COVID-19 syndrome which includes symptoms or other abnormalities present beyond 12 weeks after onset (Nalbandian et al. (2021), Nature Medicine doi.org/10.1038/s41591-021-01283-z).

Long COVID may be independent of acute illness severity. For example, it has been reported that 34-54% of patients with mild COVID-19 during the acute phase have persistent symptoms after 2-4 months and more than a quarter report developing a new neurological symptom after acute illness (Salmon-Ceron et al. (2021), J. Infect 82: e1-e4). In addition, as many as three-quarters of patients that were hospitalized with COVID-19 have at least one ongoing symptom six months after acute illness with the most reported symptoms being fatigue and muscle weakness (Huang et al. (2011), Lancet 397(10270): 220-232).

There is a need in the art for therapeutic interventions for patients suffering from Long COVID.

### SUMMARY OF THE INVENTION

The invention provides methods of treating long COVID in a patient in need thereof, comprising administering to said patient a composition comprising a therapeutically effective amount of at least one human milk oligosaccharides, wherein such treatment reduces or improves at least one sign or symptom of long COVID.

The composition comprises an effective amount of the one or more human milk oligosaccharides. In certain aspects, the composition comprises one or more HMOs selected from lacto-N-tetraose (LNT), lacto-N-neotetraose (LNnT), lacto-N-hexaose (LNH), lacto-N-neohexaose (LNnH), 2′fucosyllactose (2′FL), 3′fucosyllacose (3′FL), lacto-difucotetraose (LDFT), lacto-N-fucopenaose II/III (LNFP II/III), lactose-N-fucopentaose I (LNFP I), lacto-N-difuco-hexaose I (LNDFH I), lacto-N-difuco-hexaose II (LNDFH II), difucosyl-para-lacto-N-neohexaose (DFpLNnH), difucosyllacto-N-hexaose c (DFLNH c), 3′sialyllactose (3′SL), 6′sialyllactose (6′SL), LS-tetrasaccharide a (LSTa), LS-tetrasaccharide b (LST b), LS-tetrasaccharide c (LST c), 3′-sialyl-N-acetyllactosamine (3′SLN), 6′-sialyl-N-acetyllactosamine (6′SLN), or disialyllacto-N-tetraose (DSLNT), or a combination of any of thereof. In yet other aspects, the one or more HMOs is selected from 2′FL, 3′FL, 3′SL, 6′SL, LNT, or LNnT, or a combination of any of thereof.

In yet further aspects, the composition can comprise a mixture of two, three, four or five human milk oligosaccharides. Exemplary mixtures include: 2′FL and LNT; 2′FL and LNnT; 2′FL, 3′FL, 3′SL, 6′SL and LNT; 3′SL and 6′SL; and 6′SL and LNT.

### DETAILED DESCRIPTION OF THE INVENTION

As used herein and in the appended claims, the singular forms “a,” “an,” and “the” include plural referents unless the context clearly dictates otherwise. Thus, for example, reference to “an oligosaccharide” or “an HMO” includes a plurality of such oligosaccharides and reference to “a therapeutic agent” includes reference to one or more therapeutic agents and equivalents thereof known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs.

Although many methods and reagents are similar or equivalent to those described herein, the exemplary methods and materials are disclosed herein. All publications mentioned herein are incorporated herein by reference in full for the purpose of describing and disclosing the methodologies, which might be used in connection with the description herein. Moreover, for terms expressly defined in this disclosure, the definition of the term as expressly provided in this disclosure will control in all respects, even if the term has been given a different meaning in a publication, dictionary, treatise, and the like.

The term “about” as used herein, in reference to a numerical value or range, allows for a degree of variability in the value or range, for example, within 10%, within 5%, or within 4%, or within 2% of the value or range.

The term “pharmaceutically acceptable carrier,” “pharmaceutically acceptable excipient,” “physiologically acceptable carrier,” or “physiologically acceptable excipient” as used herein, refers to a pharmaceutically-acceptable material, composition, or vehicle, such as a liquid or solid filler, diluent, excipient, solvent, or encapsulating material. Each component must be “pharmaceutically acceptable” in the sense of being compatible with the other ingredients of a pharmaceutical formulation. It must also be suitable for use in contact with the tissue or organ of humans and animals without excessive toxicity, irritation, allergic response, immunogenecity, or other problems or complications, commensurate with a reasonable benefit/risk ratio. Examples of “pharmaceutically acceptable carriers” and “pharmaceutically acceptable excipients” can be found in the following, Remington: The Science and Practice of Pharmacy, 21st Edition; Lippincott Williams & Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004.

The term “subject” as used herein, refers to an animal, including, but not limited to, a primate (e.g., human, monkey, chimpanzee, gorilla, and the like), rodents (e.g., rats, mice, gerbils, hamsters, ferrets, and the like), lagomorphs, swine (e.g., pig, miniature pig), equine, canine, feline, and the like. The terms “subject” and “patient” are used interchangeably herein. For example, a mammalian subject can refer to a human patient. In preferred aspects, the subject is a human patient.

The term “release controlling excipient” as used herein, refers to an excipient whose primary function is to modify the duration or place of release of the active substance from a dosage form as compared with a conventional immediate release dosage form. The term “non-release controlling excipient” as used herein, refers to an excipient whose primary function do not include modifying the duration or place of release of the active substance from a dosage form as compared with a conventional immediate release dosage form.

The term “substantially pure” as used herein in reference to a given oligosaccharide means that the oligosaccharide is substantially free from other biological macromolecules. The substantially pure oligosaccharide is at least 75% (e.g., at least 80, 85, 95, or 99%) pure by dry weight. Purity can be measured by any appropriate standard method, for example, by column chromatography, polyacrylamide gel electrophoresis, or HPLC analysis.

The terms “treat”, “treating” and “treatment,” as used herein, refers to ameliorating symptoms associated with a disease, condition, or disorder (e.g., one or more symptoms of Long COVID) including inhibiting the progress of the disease or disorder (e.g., one or more symptoms of Long COVID) reducing the severity of the disease or disorder (e.g., one or more symptoms of Long COVID) and/or lessening the severity or frequency of symptoms of the disease, condition, or disorder.

The present invention encompasses co-administration of the composition comprising the one or more human milk oligosaccharide and an additional active agent. As used here “co-administration” means administration of at least two therapeutically active drugs or compositions (e.g., administration of the human milk oligosaccharide and an additional active agent, such as an additional therapeutic agent or nutritional supplement, or pharmaceutical compositions thereof), at different times or simultaneously or substantially simultaneously in either separate formulation or the same formulation/composition. When the at least two therapeutic agents are administered at different times, their administration can be separated by minutes, hours, days, weeks, or months, and/or be administered as part of the same treatment regimen.

An “effective amount” or a “therapeutically effective amount” of an agent (e.g., one or more HMOs or additional active agent) as described herein refers to an amount of the active agent, alone or in combination with another active agent, that is sufficient to achieve a specific effect or result, and/or treats the disease or condition and/or the symptoms therefore, for example, alleviating, in whole or in part, symptoms associated with the disorder or condition, or halts or slows further progression or worsening of those symptoms, or prevents or provides prophylaxis for the disorder or condition. For example, an “effective amount” of a human milk oligosaccharide, as described herein, encompasses an amount that, alone or in combination with another human milk oligosaccharide, is effective to reduce or ameliorate a sign or symptom of Long COVID.

The term “therapeutically acceptable” refers to those compounds (or salts, prodrugs, tautomers, zwitterionic forms, etc.) which are suitable for use in contact with the tissues of patients without excessive toxicity, irritation, allergic response, immunogenecity, are commensurate with a reasonable benefit/risk ratio, and are effective for their intended use.

The terms “active agent,” “drug,” “therapeutic agent,” are used interchangeably herein and refer to an agent administered as part of a method of treatment, alone or in combination with one or more pharmaceutically acceptable excipients and/or carriers, to a subject for treating, preventing, or ameliorating one or more symptoms of a disorder. The terms “active agent,” “therapeutic agent,” and “drug” as used herein includes, but are not limited to, human milk oligosaccharides.

The term “disorder” as used herein is intended to be generally synonymous, and is used interchangeably with, the terms “disease,” “syndrome” and “condition” (as in medical condition), in that all reflect an abnormal condition of the body or of one of its parts that impairs normal functioning and is typically manifested by distinguishing signs and symptoms. For example, Long COVID can be considered a disease, disorder, condition, and/or a syndrome.

The terms “COVID-19” and “SARS-CoV-2 infection” are used interchangeably herein.

As used herein, “Long COVID” or “Long COVID-19” refers to post-acute COVID or sequelae of COVID-19. In the literature, this condition can also be termed “long haul COVID,” “post-acute COVID-19,” “persistent COVID-19 symptoms,” “post-COVID-19 manifestations,” “post-infection” symptoms or conditions, “long-term COVID-19 effects,” and “post-COVID-19 syndrome,” and the like. At this time, there is not a consensus definition for Long COVID but it is characterized by a symptoms, sign or abnormal clinical parameter (which can be referred to collectively herein as “a sign or symptom” or as “a symptom” or plural referents thereof) that persist after the acute phase of infection. The acute phase of infection (which can also be referred to as the “infection phase”) is the initial illness period following infection. For example, Long COVID includes symptoms experienced three or more weeks after COVID-19 onset or three or more weeks after onset or after the acute phase. In some examples, the subject or patient can suffer at least one ongoing or persistent symptom at least 30 days or more after onset or after the acute phase. In other examples, the subject or patient can suffer at least one ongoing or persistent symptom at least 60 days or more after onset or after the acute phase. Signs or symptoms of Long COVID are described in more detail below and include, but are not limited to, fatigue, hypertension, chills, pain, weight loss, sweat, headache, attention disorder, anosmia, memory loss, anxiety, depression, fever, sleep disorder, sleep apnea, mental health, psychiatric illness, dizziness, stroke, dysphoria, mood disorder, obsessive compulsive disorder (OCD), post-traumatic stress disorder (PTSD), paranoia, limb edema, joint pain, digestive disorders, cutaneous signs, renal failure, diabetes mellitus, arrythmia, myocarditis, discontinuous flushing, palpitations, chest pain discomfort, resting heart rate increase, polypnea, cough, sputum, throat pain, ageusia, red eyes, hair loss, hearing loss, tinnitus, nausea, dyspnea, pulmonary fibrosis and reduced pulmonary capacity (Lopez-Leon (2021)).

The invention encompasses methods of treating Long COVID in a subject in need thereof comprising administering to said subject a composition comprising an effective amount of one or more human milk oligosaccharide. For example, such treatment reduces or improves at least one sign or symptom of long COVID. Signs or symptoms of long COVID include, but are not limited to, brain fog or other neurological signs and symptoms (including, but not limited, memory loss, attention disorder, and delirium); depression, anxiety, or other neuropsychiatric signs and symptoms (including, but not limited to, obsessive compulsive disorder, post-traumatic stress disorder, psychosis, and paranoia); fatigue; muscle weakness; dyspnea, cough, or other respiratory symptom (including, for example, sore throat); insomnia or sleep disturbance (including, but not limited to, sleep disorder or sleep apnea); headache; paresthesias; dysautonomia; dizziness; pain; weight loss; hypertension; olfactory or gustatory dysfunction (including, for example, anosmia or ageusia); hair loss or other dermatologic symptom; palpitations/tachycardia, chest pain, or other cardiac symptom; arthralgia/joint pain; rhinitis; Sicca/Sjogren syndrome; fever; gastroenterological symptom (including, but not limited to, abdominal pain and diarrhea); gut microbiome dysbiosis; or a combination of any of thereof.

In certain aspects, the methods described herein reduce fatigue. The reduction of fatigue can be measured, for example, by visual analogue scale, Samn-Perelli seven point fatigue scale, Karolinska Sleepiness Scale, or Psychomotor Vigilance Task. In other aspects, the methods described herein reduce muscle weakness as measured by a test selected from the group consisting of lower handgrip muscle strength, short physical performance battery (SPPB) score, Timed Up and Go test (TUGT), walking speed (WS), and/or grip strength (GS).

In additional aspects, the method comprising administration of at least one HMO reduces or improves brain fog. The reduction or improvement in brain fog can be measured by cognitive battery score, for example.

In further aspects, the method reduces or improves memory impairment. The reduction or improvement in memory impairment can, for example, be measured by a test selected from Short Test of Mental Status, the Montreal Cognitive Assessment (MoCA) or the Mini-Mental State Examination (MMSE), or by an assessment of symptoms by a physician (see, for example, mayoclinic.org/diseases-conditions/mild-cognitive-impairment/diagnosis-treatment/drc-20354583; the contents of which are expressly incorporated by reference herein).

The methods described herein can additionally reduce dyspnea, for example, as measured by a clinical scale selected from the group consisting of the Medical Research Council (MRC) scale, the Baseline Dyspnea Index (BDI), and Transitional Dyspnea Index (TDI), or a combination thereof (see, for example, Crisafulli et al. (2010), Mutidiscip Respir Med 5(3): 202-210; the contents of which are expressly incorporated by reference herein).

In additional aspects, the method of the present invention improves insomnia and/or sleep disturbance. The improvement in insomnia and/or sleep disturbance can, for example, be measured by the Karolinska Sleep Scale (KSS) or the Sleepiness Symptoms Questionnaire (SSQ).

In yet additional aspects, the methods described herein reduce dyspnea. The reduction or improvement in dyspnea can be measured by a clinical scale selected from the group consisting of the Medical Research Council (MRC) scale, the Baseline Dyspnea Index (BDI), and Transitional Dyspnea Index (TDI), or a combination thereof.

In additional aspects, the method reduces or improves depression or anxiety; improves or reduces headache, disautonomias, and/or paresthesias.

The method can also be used to improves olfactory dysfunction or gustatory dysfunction. Olfactory dysfunction includes, for example, anosmia. Gustatory dysfunction includes, for example, ageusia.

The method additionally can be used to reduce hair loss or other dermatologic symptom. Non-limiting examples of dermatologic symptoms include rash and itch.

The method can also be used to reduce heart palpitations.

In additional aspects, the method is used to treat joint pain or arthralgia.

In further aspects, the method described herein improves gut microbiome dysbiosis or persistent dysbiosis. Dysbiosis has been reported as a Long COVID symptom (see, for example, doi.org/10.20944/preprints202012.0242.v1 or webmd.com/lung/news/20210113/peoples-microbiomes-might-influence-covid-19-severity-study #1; the contents of each of which is expressly incorporated by reference herein). Dysbiosis is defined by the loss or gain of bacteria that promotes health or disease (see, for example, Wilkins et al. (2019), Defining Dysbiosis for a Cluster of Chronic Diseases, Nature Briefing 9:12918; the contents of which are expressly incorporated by reference herein). In certain aspects, the dysbiosis is determined by fecal microbiome analysis such as 16S ribosomal RNA sequencing. In yet other aspects, the dysbiosis is determined or diagnosis by measuring an increased level of salivary cortisol, plasma IL-6, plasma IL-1β, plasma TNF-α, plasma c-reactive protein, MCP-1, MIP1-α, fecal calprotectin in the subject. The administration of the HMO as described herein can improve gastrointestinal symptoms associated with the dysbiosis. Additionally, the methods described herein can improve neurological, respiratory, or inflammatory symptoms associated with dysbiosis.

In certain aspects, the sign or symptom of Long COVID is a neurobehavioral abnormality associated with dysbiosis (see, for example, Maguire et al. (2018), Gut dysbiosis, leaky gut, and intestinal epithelial proliferation in neurological disorders: towards the development of a new therapeutic using amino acids, prebiotics, probiotics, and postbiotics, Reviews in the Neurosciences, 30(2); the contents of which are expressly incorporated by reference herein. The neurobehavioral abnormality can be an anxiety disorder, depression, brain fog, stress, and negatively impacted cognitive performance. The method can entail administering a neurobehavioral test to a subject and determining if neurobehavioral function is impacted and the subject is in need to treatment. Alternatively or in addition, the method comprises using a neurobehavioral test to monitor the effectiveness of the treatment.

The subject or patient can be a subject or patient that suffered from mild, moderate or severe COVID-19 during the acute phase of infection. In certain aspects, the patient had mild or moderate COVID-19 during the acute phase. In yet further aspect, the patient had mild COVID-19 during the acute phase. In further aspects, the patient was not hospitalized during the acute phase. Mild to moderate COVID-19 includes mild symptoms up to mild pneumonia. Severe COVID-19 includes symptoms of dyspnea, hypoxia, or more than 50% lung involvement on imaging (cdc.gov/coronavirus/2019-ncov/hcp/clinical-guidance-management-patients.html).

In certain aspects, the subject is female. For example, the patient can be female and suffered mild or moderate COVID-19 during the acute or early phase.

In other aspects, subject is male. For example, the patient can be male and suffered mild or moderate COVID-19 during the acute or early phase.

In further aspects, subject is 55 years old or less or 50 year old or less. For example, the patient can be 55 years old or less or 50 years or less and suffered mild or moderate COVID-19 during the acute or early phase.

In additional embodiments, the subject is 60 years or older or 70 years or older. For example, the patient can be 60 years old or older or 70 years or older and suffered mild or moderate COVID-19 during the acute or early phase.

In yet additional aspects, the patient has a body mass index ≥30 (or suffers from obesity). For example, the patient has a body mass index ≥30 and suffered mild or moderate COVID-19 during the acute or early phase.

It has been reported that patients more likely to suffer long COVID are female and/or experience at least five symptoms during the first week of illness (Sudre et al. (2021), Nat Med doi: 10.1038/s41591-021-01292-y; the contents of which are expressly incorporated by reference herein). In certain aspects, the patient is one that experiences at least five symptoms during the first week of infection. In yet additional aspects, the patient is female and experiences at least five symptoms during the first week of infection.

In yet additional aspects, the long COVID patient was prescribed antibiotics during their acute COVID infection.

In some embodiments, the patient has been vaccinated against SARS-CoV-2. In other aspects, the patient has not been vaccinated against SARS-CoV-2.

In certain aspects, the patient has been diagnosed with long COVID. A diagnosis of long COVID can comprise utilization of the Work Productivity and Activity Impairment (WPAI) Questionnaire. In additional embodiments, the method reduces and/or improves at least one sign or symptom of long COVID as assessed by a method comprising use of the Work Productivity and Activity Impairment (WPAI) Questionnaire. In yet additional aspects, the method reduces and/or improves at least one sign or symptom of COVID as reported by the patient or subject.

The invention comprises administration of an effective amount of one or more HMOs. In specific examples, the one or more HMO is administered in amount from about 1 to about 15 g (total HMOs) once or twice a day for one or more days, for one or more weeks, for one or more months. In another example, the one or more HMO is administered in an amount from about 1.5 to about 7.5 g twice a day, once or twice a day for one or more days, for one or more weeks, for one or more months.

An “oligosaccharide” is a saccharide polymer containing a small number (typically three to ten) of simple sugars (monosaccharides). A “human milk oligosaccharide” is an oligosaccharide found in human milk. As used herein, the term “human milk oligosaccharide” includes natural or native oligosaccharides found in human milk, as well as pharmaceutically acceptable salts, derivatives, prodrugs, and solvates thereof. The term “natural human milk oligosaccharide” or “natural HMO” refers to human milk oligosaccharides naturally found in human milk. Natural human milk oligosaccharides (HMOs) are separated into different classes including, for example, sialylated human milk oligosaccharides and fucosylated oligosaccharides. HMOs include natural sialylated human milk oligosaccharides and fucosylated oligosaccharides, as well as non-naturally occurring derivatives thereof. Natural human milk oligosaccharides (HMOs) are separated into different classes including, for example, sialylated human milk oligosaccharides (which include sialyllactoses; sialyllactoses are sialylated oligosaccharides that comprise a lactose) and fucosylated oligosaccharides (which include “fucosyllactoses”; fucosyllactoses are fucosylated oligosaccharides that comprise a lactose). HMOs include natural sialylated human milk oligosaccharides and fucosylated oligosaccharides, as well as non-naturally occurring derivatives thereof. Non-limiting examples of sialyllactoses are 3′-SL and 6′-SL. A non-limiting example of a Fucosyllactose is 2′-FL. In certain aspects, the oligosaccharides, human milk oligosaccharides or natural human milk oligosaccharides of the present invention are not isolated from human milk. The oligosaccharides of the present invention are optionally synthesized chemically, enzymatically or with synthetic biology technology, i.e. genetically engineered microorganism.

Sialyllactose is a class of human milk oligosaccharides (HMOs) that appear in two different forms in human milk. These two forms are 3′-sialyllactose (3′-SL) and 6′-sialyllactose (6′-SL):

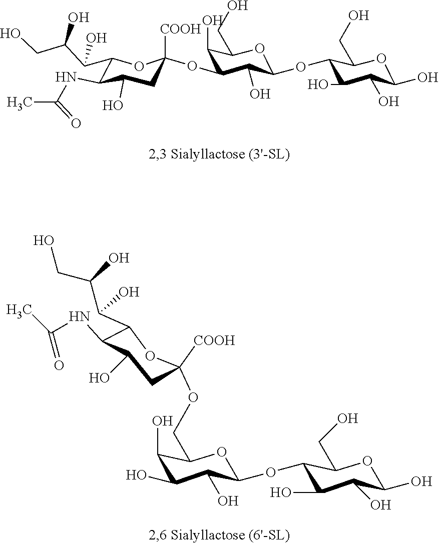

The terms “3′-SL” and “3′SL” are used interchangeably herein. Similarly, the terms “6′-SL” and “6′SL” are used interchangeably herein. Sialyllactoses have been shown to modulate acute and chronic immune responses in both murine and human derived macrophages stimulated with LPS and various pro-inflammatory cytokines. Both 3′SL and 6′SL have shown reductions in interleukin (IL)-1β, IL-2, IL-4, IL-6, IL-12, interferon (IFN) γ or TNF-α in vitro, with 3′-SL exhibiting more significant reductions. In addition, 3′SL has been shown to reduce other key target proteins, including PDL1, COX2 and select chemokines, such as CCL2 (also known as monocyte chemoattractant protein 1 (MCP1)) and CCL5. In vivo data in mouse models of rheumatoid arthritis, which include an LPS challenge, sialyllactose has shown benefit in clinical assessments of disease when administered orally.

Fucosylated oligosaccharides are a class of human milk oligosaccharides (HMOs) that have been associated with the production of anti-inflammatory short-chain fatty acids. Fucosylated oligosaccharides include, for example, 2′-fucosyllactose, 3-fucosyllactose, difucosyllactose, lacto-N-fucopentaoses (that is to say lacto-N-fucopentaose I, lacto-N-fucopentaose II, lacto-N-fucopentaose III and lacto-N-fucopentaose V), lacto-N-difucohexaose I, fucosyllacto-N-hexaose, Difucosyllacto-N-hexaose I and Difucosyllacto-N-neohexaose II. In certain aspects, the fucosylated non-digestible oligosaccharide is 2′-fucosyllactose (2′-FL). In certain aspects, the fucosylated oligosaccharide is 2′-fucosyllactose (2′-FL), 3-fucosyllactose (3′-FL), difucosyllactose (DFL). In yet further aspects, the fucosylated oligosaccharide is 2′-FL. As used herein, a “fucosylated oligosaccharide” is an oligosaccharide having the three sugar unit backbone, wherein each of the sugar units (fucose (Fuc), galactose (Gal), and glucose (Glc)) can be independently either in its native form or in a modified form. For example, the modified form of a sugar unit can be a sugar unit, in which at least one or more (e.g., 1, 2, 3, or more) of the hydroxyl groups is replaced with hydrogen, alkyl or a functional group; such as, for example, hydrogen, substituted or unsubstituted C1-C6 alkyl (e.g., methyl, ethyl), or substituted or unsubstituted amine group.

Fucosyllactose (FL) is a fucosylated non-digestible oligosaccharide present in human milk but not in cow milk. The primary fucosylated HMO is 2′-fuscosyllactose or 2′FL. It consists of three monosaccharide units, fucose, galactose and glucose linked together. Lactose is a galactose unit linked to a glucose unit via a beta 1,4 linkage. A fucose unit is linked to a galactose unit of a lactose molecule via an alpha 1,2 linkage (2′-fucosyllactose, 2′-FL) or via an alpha 1,3 linkage to the glucose unit of a lactose (3-Fucosyllactose, 3-FL). 2-′FL has the chemical structure shown below:

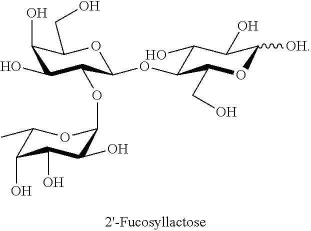

The terms 2′-fucosyllactose or “2′-FL” and “2′FL” are used interchangeably herein. 2′-fucosyllactose has been granted generally regarded as safe (GRAS) status in the U.S. and is regarded by the Europe Food Safety Authority as safe for infant and follow-on formula. 2′-FL has been shown to have many beneficial properties, such as improving of gut health through modulation of the gut microbiome as well as reduction of local gut inflammation in models of necrotizing enterocolitis and other inflammatory bowel diseases. In addition, 2′-FL has been shown to have positive effects on gut epithelial barrier function and also independent anti-inflammatory effects through the reduction in TNFα and IL-8.

Derivatives of natural HMOs can be chemically modified as compared to the natural HMO. In certain aspects, the derivative of the natural HMO retains at least 50%, at least 60%, at least 70% or more (including, e.g., at least 80%, at least 90%, at least 95%, at least 98%, at least 99% and up to 100%) of the biological functions of a natural HMO.

HMOs include, but are not limited to, compounds having a structure of Formula I, I(a), I(b), I(c), I(d), I(e), II, II(a) or IIIa:

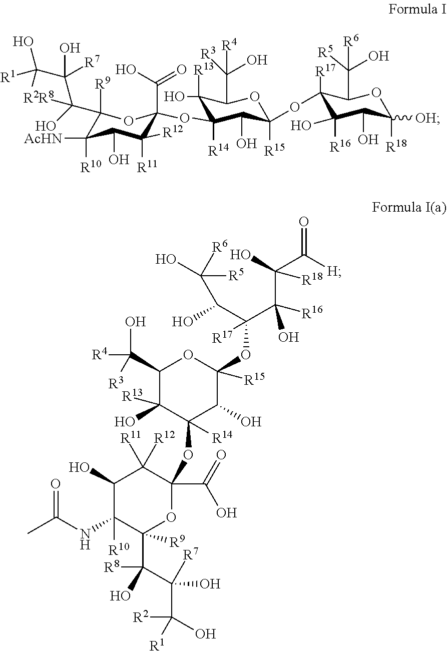

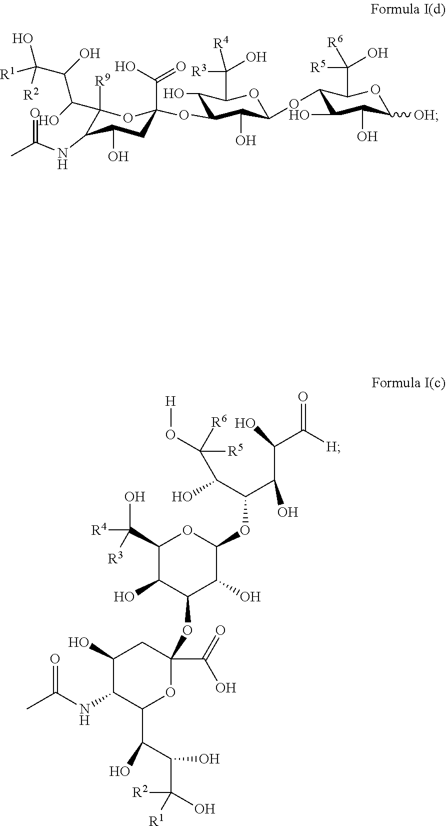

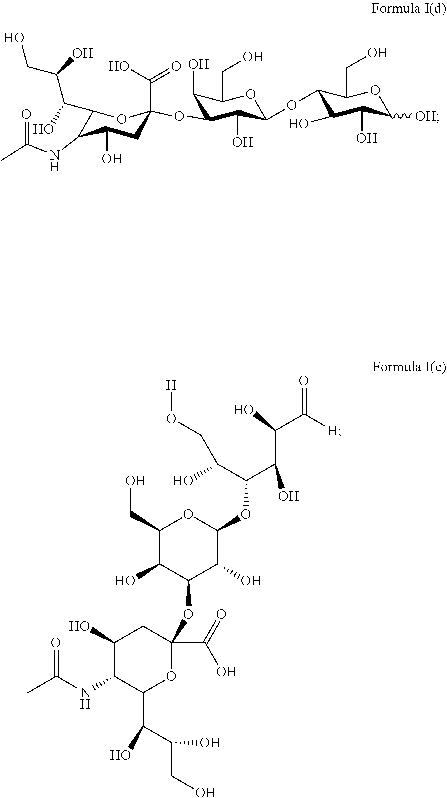

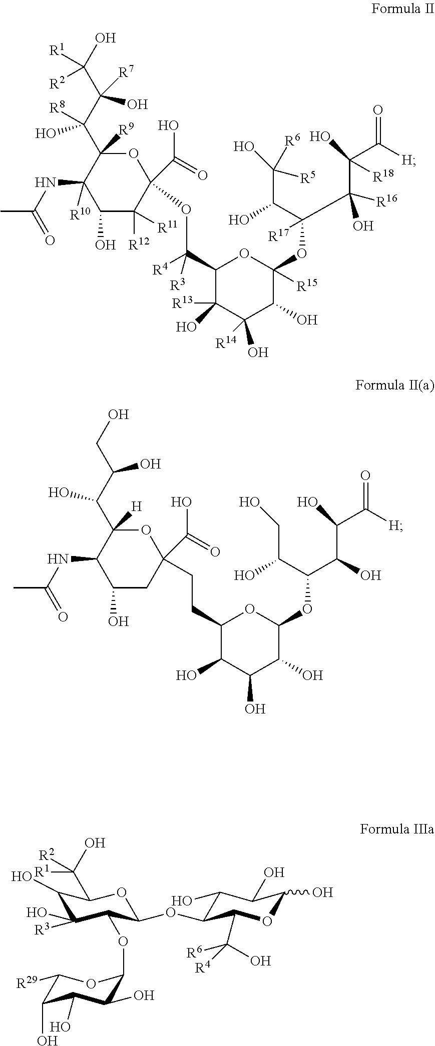

or a pharmaceutically acceptable salt, solvate, or prodrug thereof, wherein:
R¹-R¹⁸ are each independently selected from H, D, a halo, an unsubstituted or substituted (C₁-C₆)alkyl, an unsubstituted or substituted (C₁-C₆)heteroalkyl, an unsubstituted or substituted (C₂-C₆)alkenyl, an unsubstituted or substituted (C₂-C₆)heteroalkenyl, an unsubstituted or substituted (C₃-C₆)alkynyl, an unsubstituted or substituted (C₃-C₆)heteroalkynyl, an unsubstituted or substituted (C₄-C₈)cycloalkyl, an unsubstituted or substituted heterocycle, an unsubstituted or substituted aryl, —ROR′, —RN(R′)₂, —RSSR′, —SH, —RSOR′, —RSO₂R′, —RSO₂H, —RSO₃H, —RC(═S)—R′, —ROH, —RC(═O)R′, —RNO₂, —RSR′, —RCN, —RNC, —RNNR′, —RC(═O)OR′, —ROC(═O)R′, —RC(═O)H, —RC(═O)OH, —RC(═O)N(R′)₂, —RN₃, —ROCN, —RNCO, —RONO₂, —RNO, —ROP(═O)(OH)₂, and —RB(OH)₂;
R is absent or a (C₁-C₅)alkyl;
R′ is independently selected from H, D, an unsubstituted or substituted (C₁-C₆) alkyl, an unsubstituted or substituted (C₁-C₆)heteroalkyl, an unsubstituted or substituted (C₂-C₆) alkenyl, an unsubstituted or substituted (C₂-C₆)heteroalkenyl, an unsubstituted or substituted (C₃-C₆)alkynyl, an unsubstituted or substituted (C₃-C₆)heteroalkynyl, an unsubstituted or substituted (C₄-C₈)cycloalkyl, an unsubstituted or substituted heterocycle, and an unsubstituted or substituted aryl; and
R²⁹ is an unsubstituted or substituted (C₁-C₆)alkyl.

In yet additional aspects, the HMO has the Formula IIIb:

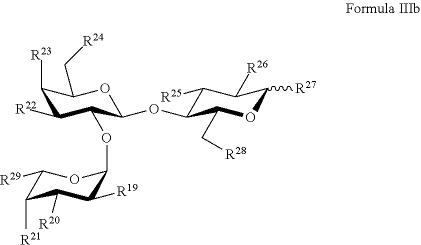

wherein:
one, two or three of R¹⁹-R²⁸ are each independently selected from the group consisting of hydrogen, an unsubstituted or substituted C₁-C₆ alkyl (including, but not limited to, methyl and ethyl) and N(R′)2 (wherein R′ is as defined above), the remainder or R₁₉-R₂₈ are —OH, and R²⁹ is substituted or unsubstituted C₁-C⁶ alkyl; or
one, two or three of R¹⁹-R²⁹ are each independently selected from NHC(O)R″, wherein R″ is unsubstituted or substituted (C₁-C₆) alkyl (including, but not limited to, methyl), the remainder or R₁₉-R₂₈ are —OH, and R²⁹ is substituted or unsubstituted C₁-C⁶ alkyl. In certain aspects, R²⁶ is NHC(O)CH₃ and R¹⁹-R²⁵ and R²⁷-R²⁸ are —OH, and R²⁹ is methyl.

The term “alkyl” refers to an organic group that is comprised of carbon and hydrogen atoms that contains single covalent bonds between carbons. Typically, an “alkyl” as used in this disclosure, refers to an organic group that contains 1, 2, 3, 4, 5, 6, 7, 8, 9, 10, 11, 12, 13, 14, 15, 16, 17, 18, 19, 20, or 30 carbon atoms, or any range of carbon atoms between or including any two of the foregoing values. Where if there is more than 1 carbon, the carbons may be connected in a linear manner, or alternatively if there are more than 2 carbons then the carbons may also be linked in a branched fashion so that the parent chain contains one or more secondary, tertiary, or quaternary carbons. An alkyl may be substituted or unsubstituted, unless stated otherwise.

The term “alkenyl” refers to an organic group that is comprised of carbon and hydrogen atoms that contains at least one double covalent bond between two carbons. Typically, an “alkenyl” as used in this disclosure, refers to organic group that contains 2, 3, 4, 5, 6, 7, 8, 9, 10, 11, 12, 13, 14, 15, 16, 17, 18, 19, 20, or 30 carbon atoms, or any range of carbon atoms between or including any two of the foregoing values. While a C2-alkenyl can form a double bond to a carbon of a parent chain, an alkenyl group of three or more carbons can contain more than one double bond. In certain instances, the alkenyl group will be conjugated, in other cases an alkenyl group will not be conjugated, and yet other cases the alkenyl group may have stretches of conjugation and stretches of non-conjugation. Additionally, if there is more than 2 carbons, the carbons may be connected in a linear manner, or alternatively if there are more than 3 carbons then the carbons may also be linked in a branched fashion so that the parent chain contains one or more secondary, tertiary, or quaternary carbons. An alkenyl may be substituted or unsubstituted, unless stated otherwise.

The term “alkynyl”, refers to an organic group that is comprised of carbon and hydrogen atoms that contains a triple covalent bond between two carbons. Typically, an “alkynyl” as used in this disclosure, refers to organic group that contains that contains 2, 3, 4, 5, 6, 7, 8, 9, 10, 11, 12, 13, 14, 15, 16, 17, 18, 19, 20, or 30 carbon atoms, or any range of carbon atoms between or including any two of the foregoing values. While a C2-alkynyl can form a triple bond to a carbon of a parent chain, an alkynyl group of three or more carbons can contain more than one triple bond.

Where if there is more than 3 carbon, the carbons may be connected in a linear manner, or alternatively if there are more than 4 carbons then the carbons may also be linked in a branched fashion so that the parent chain contains one or more secondary, tertiary, or quaternary carbons. An alkynyl may be substituted or unsubstituted, unless stated otherwise.

The term “aryl”, as used in this disclosure, refers to a conjugated planar ring system with delocalized pi electron clouds that contain only carbon as ring atoms. An “aryl” for the purposes of this disclosure encompass from 1 to 4 aryl rings wherein when the aryl is greater than 1 ring the aryl rings are joined so that they are linked, fused, or a combination thereof. An aryl may be substituted or unsubstituted, or in the case of more than one aryl ring, one or more rings may be unsubstituted, one or more rings may be substituted, or a combination thereof.

The term “cycloalkyl”, as used in this disclosure, refers to an alkyl that contains at least 3 carbon atoms but no more than 12 carbon atoms connected so that it forms a ring. A “cycloalkyl” for the purposes of this disclosure encompasses from 1 to 4 cycloalkyl rings, wherein when the cycloalkyl is greater than 1 ring, then the cycloalkyl rings are joined so that they are linked, fused, or a combination thereof. A cycloalkyl may be substituted or unsubstituted, or in the case of more than one cycloalkyl ring, one or more rings may be unsubstituted, one or more rings may be substituted, or a combination thereof.

The term “hetero-” when used as a prefix, such as, hetero-alkyl, hetero-alkenyl, hetero-alkynyl, or hetero-hydrocarbon, for the purpose of this disclosure refers to the specified hydrocarbon having one or more carbon atoms replaced by non-carbon atoms as part of the parent chain. Examples of such non-carbon atoms include, but are not limited to, N, O, S, Si, Al, B, and P. If there is more than one non-carbon atom in the hetero-based parent chain then this atom may be the same element or may be a combination of different elements, such as N and O. In a particular embodiment, a “hetero”-hydrocarbon (e.g., alkyl, alkenyl, alkynyl) refers to a hydrocarbon that has from 1 to 3 C, N and/or S atoms as part of the parent chain.

The term “heterocycle,” as used herein, refers to ring structures that contain at least 1 noncarbon ring atom. A “heterocycle” for the purposes of this disclosure encompass from 1 to 4 heterocycle rings, wherein when the heterocycle is greater than 1 ring the heterocycle rings are joined so that they are linked, fused, or a combination thereof. A heterocycle may be aromatic or nonaromatic, or in the case of more than one heterocycle ring, one or more rings may be nonaromatic, one or more rings may be aromatic, or a combination thereof. A heterocycle may be substituted or unsubstituted, or in the case of more than one heterocycle ring one or more rings may be unsubstituted, one or more rings may be substituted, or a combination thereof. Typically, the noncarbon ring atom is N, O, S, Si, Al, B, or P. In the case where there is more than one noncarbon ring atom, these noncarbon ring atoms can either be the same element, or combination of different elements, such as N and O.

Examples of heterocycles include, but are not limited to: a monocyclic heterocycle such as, aziridine, oxirane, thiirane, azetidine, oxetane, thietane, pyrrolidine, pyrroline, imidazolidine, pyrazolidine, pyrazoline, dioxolane, sulfolane 2,3-dihydrofuran, 2,5-dihydrofuran tetrahydrofuran, thiophane, piperidine, 1,2,3,6-tetrahydro-pyridine, piperazine, morpholine, thiomorpholine, pyran, thiopyran, 2,3-dihydropyran, tetrahydropyran, 1,4-dihydropyridine, 1,4-dioxane, 1,3-dioxane, dioxane, homopiperidine, 2,3,4,7-tetrahydro-1H-azepine homopiperazine, 1,3-dioxepane, 4,7-dihydro-1,3-dioxepin, and hexamethylene oxide; and polycyclic heterocycles such as, indole, indoline, isoindoline, quinoline, tetrahydroquinoline, isoquinoline, tetrahydroisoquinoline, 1,4-benzodioxan, coumarin, dihydrocoumarin, benzofuran, 2,3-dihydrobenzofuran, isobenzofuran, chromene, chroman, isochroman, xanthene, phenoxathiin, thianthrene, indolizine, isoindole, indazole, purine, phthalazine, naphthyridine, quinoxaline, quinazoline, cinnoline, pteridine, phenanthridine, perimidine, phenanthroline, phenazine, phenothiazine, phenoxazine, 1,2-benzisoxazole, benzothiophene, benzoxazole, benzthiazole, benzimidazole, benztriazole, thioxanthine, carbazole, carboline, acridine, pyrolizidine, and quinolizidine. In addition to the polycyclic heterocycles described above, heterocycle includes polycyclic heterocycles wherein the ring fusion between two or more rings includes more than one bond common to both rings and more than two atoms common to both rings. Examples of such bridged heterocycles include quinuclidine, diazabicyclo[2.2.1]heptane and 7-oxabicyclo[2.2.1]heptane.

The terms “heterocyclic group”, “heterocyclic moiety”, “heterocyclic”, or “heterocyclo” used alone or as a suffix or prefix, refers to a heterocycle that has had one or more hydrogens removed there from.

The term “hydrocarbons” refers to groups of atoms that contain only carbon and hydrogen. Examples of hydrocarbons that can be used in this disclosure include, but are not limited to, alkanes, alkenes, alkynes, arenes, and benzyls.

The term “optionally substituted” means independent replacement of one or more hydrogen atoms with a substituent. The term “optionally substituted” also refers to a functional group, typically a hydrocarbon or heterocycle, where one or more hydrogen atoms may be replaced with a substituent. Accordingly, “optionally substituted” refers to a functional group that is substituted, in that one or more hydrogen atoms are replaced with a substituent, or unsubstituted, in that the hydrogen atoms are not replaced with a substituent. For example, an optionally substituted hydrocarbon group refers to an unsubstituted hydrocarbon group or a substituted hydrocarbon group.

As described herein, the subject is administered a composition comprising one or more human milk oligosaccharides. The composition can comprise 10% or more, 20% or more, 30% or more, 40% or more, or 50% or more by mass one or more human milk oligosaccharide.

In certain aspects, the composition is not human milk. In additional aspects, the composition is not derived from human milk.

In certain aspects, the one or more human milk oligosaccharides are selected from lacto-N-tetraose (LNT), lacto-N-neotetraose (LNnT), lacto-N-hexaose (LNH), lacto-N-neohexaose (LNnH), 2′fucosyllactose (2′FL), 3′fucosyllacose (3′FL), lacto-difucotetraose (LDFT), lacto-N-fucopenaose II/III (LNFP II/III), lactose-N-fucopentaose I (LNFP I), lacto-N-difuco-hexaose I (LNDFH I), lacto-N-difuco-hexaose II (LNDFH II), difucosyl-para-lacto-N-neohexaose (DFpLNnH), difucosyllacto-N-hexaose c (DFLNH c), 3′sialyllactose (3′SL), 6′sialyllactose (6′SL), LS-tetrasaccharide a (LSTa), LS-tetrasaccharide b (LST b), LS-tetrasaccharide c (LST c), 3′-sialyl-N-acetyllactosamine (3′SLN), 6′-sialyl-N-acetyllactosamine (6′SLN), or disialyllacto-N-tetraose (DSLNT), or a combination of any of thereof. In yet other aspects, the one or more human milk oligosaccharides are selected from 2′FL, 3′FL, 3′SL, 6′SL, LNT, or LNnT, or a combination of any of thereof.

The composition administered to the subject can comprise one HMO or can comprise a mixture of two, three, four, five or more HMOs. In certain aspects, the composition comprises one HMO and the HMO is selected from the group consisting of 2′FL, 3′FL, 3′SL, 6′SL, LNT, or LNnT.

In further aspects, the composition comprises a mixture of 2′FL and at least one other HMO. In certain aspects, the composition comprises 2′FL and LNT; 2′FL and LNnT; 2′FL, 3′FL, 3′SL, 6′SL and LNT. The composition comprising 2′FL and LNT includes a 4:1 mixture of 2′FL and LNT; such a composition is GRAS (generally regarded as safe) and is available from Glycom, Lyngby, Denmark. A composition comprising 2′FL, 3′FL, 3′SL, 6′SL and LNT is sold by Jennewein Biotechnologie and is GRAS.

In further aspects, the composition comprises a mixture of one neutral core and one neutral fucosylated human milk oligosaccharide. In additional embodiments, the composition comprises a mixture of one neutral and one acidic human milk oligosaccharide.

The composition can be administered orally. The composition can be formulated as a liquid formulation (e.g., aqueous solutions), a powder, a nutritional additive, protein bar, as well as a tablet and capsule.

The oligosaccharides described herein can be synthesized chemically, enzymatically or with synthetic biology technology, i.e. genetically engineered microorganism. Human milk oligosaccharides, including 2′-fucosyllactose, 3′sialyllactose, and 6′sialyllactose, can, for example, be readily prepared with well-established synthetic biology methods.

In some embodiments, the composition comprises at least 9% (e.g., 10, 15, 20, 25, 30, 35, 40, 45, 50, 55, 60, 65, 70, 75, 80, 85, 90, 95, or 100%; or any value between any of the foregoing) of the total human milk oligosaccharides in the composition.

In certain aspects, the pharmaceutical composition one or more human milk oligosaccharides, and optionally a pharmaceutically acceptable carrier or excipient.

The oligosaccharide can be administered in an amount from about 1 g to about 20 g, about 1 to about 15 g, or about 2.5 to 7.5 g per day or per dose. The oligosaccharide can be administered at various intervals, for example, once a day, twice a day, three times a day, once a week, twice a week, or as needed.

In further aspects, the methods additionally provide an improvement in the subject's microbiota (gut and/or oral) composition. Improvement in, or avoidance of, gastrointestinal symptoms, such as constipation, diarrhea, stool consistency, stool smell, flatulence and abdominal pain is desirable, such as, for example, at weeks 8 and 16 of the beginning of treatment.

Oral administration of the oligosaccharides of the disclosure provide for systemic circulation of the oligosaccharides both in infants and adults. Unlike other drug products approved by the FDA, the oligosaccharides described herein can not only be administered to treat a disease or disorder in an adult subject, but can also be administered to pregnant females, infants, and subjects who have impaired organ function (e.g., liver disfunction, kidney failure). Due to the oligosaccharides of the disclosure having little to no adverse effects in humans, this form of therapy could be used as a preventive, as a first line therapy option, or as an adjunct to existing therapies that would be well tolerated by patients of either sex.

In a further embodiment, said oligosaccharide is substantially a single enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, substantially an individual diastereomer, or a mixture of about 90% or more by weight of an individual diastereomer and about 10% or less by weight of any other diastereomer.

The oligosaccharides disclosed herein may be enantiomerically pure, such as a single enantiomer or a single diastereomer, or be stereoisomeric mixtures, such as a mixture of enantiomers, a racemic mixture, or a diastereomeric mixture. As such, one of skill in the art will recognize that administration of an oligosaccharide in its (R) form is equivalent, for oligosaccharides that undergo epimerization in vivo, to administration of the oligosaccharide in its (S) form. Conventional techniques for the preparation/isolation of individual enantiomers include chiral synthesis from a suitable optically pure precursor or resolution of the racemate using, for example, chiral chromatography, recrystallization, resolution, diastereomeric salt formation, or derivatization into diastereomeric adducts followed by separation.

When the oligosaccharide disclosed herein contains an acidic or basic moiety, it may also be disclosed as a pharmaceutically acceptable salt (See, Berge et al., J. Pharm. Sci. 1977, 66, 1-19; and “Handbook of Pharmaceutical Salts, Properties, and Use,” Stah and Wermuth, Ed.; Wiley-VCH and VHCA, Zurich, 2002).

Suitable acids for use in the preparation of pharmaceutically acceptable salts include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxo-glutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for use in the preparation of pharmaceutically acceptable salts, including, but not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, secondary amines, triethanolamine, trimethylamine, triethylamine, N-methyl-D-glucamine, 2-amino-2-(hydroxymethyl)-1,3-propanediol, and tromethamine.

The oligosaccharide as disclosed herein may also be designed as a prodrug, which is a functional derivative of the oligosaccharide as disclosed herein and is readily convertible into the parent oligosaccharide in vivo. Prodrugs are often useful because, in some situations, they may be easier to administer than the parent oligosaccharide. They may, for instance, be bioavailable by oral administration whereas the parent oligosaccharide is not.

The prodrug may also have enhanced solubility in pharmaceutical compositions over the parent oligosaccharide. A prodrug may be converted into the parent drug by various mechanisms, including enzymatic processes and metabolic hydrolysis. See Harper, Progress in Drug Research 1962, 4, 221-294; Morozowich et al. in “Design of Biopharmaceutical Properties through Prodrugs and Analogs,” Roche Ed., APHA Acad. Pharm. Sci. 1977; “Bioreversible Carriers in Drug in Drug Design, Theory and Application,” Roche Ed., APHA Acad. Pharm. Sci. 1987; “Design of Prodrugs,” Bundgaard, Elsevier, 1985; Wang et al., Curr. Pharm. Design 1999, 5, 265-287; Pauletti et al., Adv. Drug. Delivery Rev. 1997, 27, 235-256; Mizen et al., Pharm. Biotech. 1998, 11, 345-365; Gaignault et al., Pract. Med. Chem. 1996, 671-696; Asgharnejad in “Transport Processes in Pharmaceutical Systems,” Amidon et al., Ed., Marcell Dekker, 185-218, 2000; Balant et al., Eur. J. Drug Metab. Pharmacokinet. 1990, 15, 143-53; Balimane and Sinko, Adv. Drug Delivery Rev. 1999, 39, 183-209; Browne, Clin. Neuropharmacol. 1997, 20, 1-12; Bundgaard, Arch. Pharm. Chem. 1979, 86, 1-39; Bundgaard, Controlled Drug Delivery 1987, 17, 179-96; Bundgaard, Adv. Drug Delivery Rev. 1992, 8, 1-38; Fleisher et al., Adv. Drug Delivery Rev. 1996, 19, 115-130; Fleisher et al., Methods Enzymol. 1985, 112, 360-381; Farquhar et al., J. Pharm. Sci. 1983, 72,324-325; Freeman et al., J. Chem. Soc., Chem. Commun. 1991, 875-877; Friis and Bundgaard, Eur. J. Pharm. Sci. 1996, 4, 49-59; Gangwar et al., Des. Biopharm. Prop. Prodrugs Analogs, 1977, 409-421; Nathwani and Wood, Drugs 1993, 45, 866-94; Sinhababu and Thakker, Adv. Drug Delivery Rev. 1996, 19, 241-273; Stella et al., Drugs 1985, 29, 455-73; Tan et al., Adv. Drug Delivery Rev. 1999, 39, 117-151; Taylor, Adv. Drug Delivery Rev. 1996, 19, 131-148; Valentino and Borchardt, Drug Discovery Today 1997, 2, 148-155; Wiebe and Knaus, Adv. Drug Delivery Rev. 1999, 39, 63-80; Waller et al., Br. J. Clin. Pharmac. 1989, 28, 497-507.

The oligosaccharide may be produced by biotechnological means using enzyme-based fermentation technology (recombinant or natural enzymes) or microbial fermentation technology. In the latter case, microbes may either express their natural enzymes and substrates or may be engineered to produce respective substrates and enzymes. Single microbial cultures and/or mixed cultures may be used. Alternatively, the oligosaccharides may be produced by chemical synthesis from lactose and other substrates. Biotechnological approaches have made it possible for the large scale, cost-efficient production of target oligosaccharides.

Precisely, the oligosaccharides disclosed herein can be produced in high yields in aqueous media by fermentation of genetically modified bacteria, yeasts or other microorganisms. See, for example, WO200104341; WO2007101862, WO2010070104; WO2010142305; WO2012112777; Priem et al., Glycobiology 12:235 (2002); Drouillard et al., Angew. Chem. Int. Ed. 45:1778 (2006); Han et al., Biotechnol. Adv. 30:1268 (2012); Lee et al., Microb. Cell Fact. 11:48 (2012); Baumgartner et al., Microb. Cell Fact. 12:40 (2013); and WO2014135167A1.

Alternatively, the oligosaccharides of the disclosure can be synthesized based upon methods described in WO2011100980A1; WO2012007588A1; WO2012127410A1; WO2012155916A1; WO2013044928A1; and U.S. Pat. No. 9,102,966B2. 2′-FL can be made as described in WO2010/115934 and WO2010/115935, 3-FL can be made as described in WO2013/139344. Fucosylated oligosaccharides can be made as described in WO2012/127410. With regard to biotechnological methods, WO2001/04341 and WO2007/101862 describe how to make oligosaccharides optionally substituted by fucose using genetically modified E. coli. The oligosaccharides disclosed herein can be produced in high yields in aqueous media by fermentation of genetically modified bacteria, yeasts or other microorganisms. See, for example, WO200104341; WO2007101862, WO2010070104; WO2010142305; WO2012112777; Priem et al., Glycobiology 12:235 (2002); Drouillard et al., Angew. Chem. Int. Ed. 45:1778 (2006); Han et al., Biotechnol. Adv. 30:1268 (2012); Lee et al., Microb. Cell Fact. 11:48 (2012); Baumgartner et al., Microb. Cell Fact. 12:40 (2013); and WO2014135167A1.

Alternatively, the oligosaccharides of the disclosure can be synthesized based upon methods described in WO2011100980A1; WO2012007588A1; WO2012127410A1; WO2012155916A1; WO2013044928A1; and U.S. Pat. No. 9,102,966B2. 2′-FL can be made as described in WO2010/115934 and WO2010/115935, 3-FL can be made as described in WO2013/139344, 6′-SL and salts thereof can be made as described in WO2010/100979, sialylated oligosaccharides can be made as described in WO2012/113404 and mixtures of human milk oligosaccharides can be made as described in WO 2012/113405. As examples of enzymatic production, sialylated oligosaccharides can be made as described in WO2012/007588, fucosylated oligosaccharides can be made as described in WO2012/127410.

With regards to biotechnological methods, WO2001/04341 and WO2007/101862 describe how to make oligosaccharides optionally substituted by fucose or sialic acid using genetically modified E. coli.

In a certain embodiment, the composition described herein can further comprise one or more foodgrade agents. Examples of foodgrade agents that can be used with the oligosaccharides disclosed herein, include, but are not limited to, milk (e.g., cow's milk, almond milk, soy milk), yogurt, maltodextrin, milk protein concentrate, Sucromalt, glycerine, cocoa powder, soy protein isolate, fructose, vegetable or animal oils (e.g., high oleic safflower oil, soy oil, canola oil), plant sterol esters, HMSs/HMOs, soy lecithin, carrageenan, taurine, L-carnitine, vitamins and/or minerals (e.g., sodium ascorbate, potassium citrate, sodium phosphate, calcium citrate, choline chloride, potassium chloride, sodium citrate, magnesium oxide, alpha-tocopheryl acetate, zinc sulfate, ferrous sulfate, niacinamide, calcium pantothenate, vitamin A palmitate, citric acid, manganese sulfate, pyridoxine hydrochloride, vitamin D3, copper sulfate, thiamine mononitrate, riboflavin, beta carotene, folic acid, biotin, potassium iodide, chromium chloride, sodium selenate, sodium molybdate, phytonadione, vitamin B12, magnesium chloride, calciumphosphate).

Disclosed herein are methods of treatment comprising administering pharmaceutical compositions comprising one or more oligosaccharides described herein, or a pharmaceutically acceptable salt, solvate, or prodrug thereof, as an active ingredient, combined with a pharmaceutically acceptable vehicle, carrier, diluent, or excipient, or a mixture thereof, in combination with one or more pharmaceutically acceptable excipients or carriers.

Disclosed herein are pharmaceutical compositions in modified release dosage forms, which comprise one or more oligosaccharides of the disclosure, or a pharmaceutically acceptable salt, solvate, or prodrug thereof, and one or more release controlling excipients or carriers as described herein. Suitable modified release dosage vehicles include, but are not limited to, hydrophilic or hydrophobic matrix devices, water-soluble separating layer coatings, enteric coatings, osmotic devices, multiparticulate devices, and combinations thereof.

The pharmaceutical compositions may also comprise non-release controlling excipients or carriers.

Further disclosed herein are pharmaceutical compositions in enteric coated dosage forms, which comprise one or more human milk oligosaccharides as disclosed herein, or a pharmaceutically acceptable salt, solvate, or prodrug thereof, and one or more release controlling excipients or carriers for use in an enteric coated dosage form. The pharmaceutical compositions may also comprise non-release controlling excipients or carriers.

Further disclosed herein are pharmaceutical compositions in effervescent dosage forms, which comprise one or more human milk oligosaccharides as disclosed herein in substantially pure form (e.g., lacking other oligosaccharides found in milk), or a pharmaceutically acceptable salt, solvate, or prodrug thereof, and one or more release controlling excipients or carriers for use in an effervescent dosage form. The pharmaceutical compositions may also comprise non-release controlling excipients or carriers.

Additionally, disclosed are pharmaceutical compositions in a dosage form that has an instant releasing component and at least one delayed releasing component, and is capable of giving a discontinuous release of one or more human milk oligosaccharides disclosed herein in the form of at least two consecutive pulses separated in time (e.g., separated in time from 0.1 up to 24 hours or a few days). The pharmaceutical compositions comprise an oligosaccharide as disclosed herein, or a pharmaceutically acceptable salt, solvate, or prodrug thereof, and one or more release controlling and non-release controlling excipients or carriers, such as those excipients or carriers suitable for a disruptable semi-permeable membrane and as swellable substances.

Disclosed herein also are pharmaceutical compositions in a dosage form for oral administration to a subject, which comprise one or more as disclosed herein, or a pharmaceutically acceptable salt, solvate, or prodrug thereof, and one or more pharmaceutically acceptable excipients or carriers, enclosed in an intermediate reactive layer comprising a gastric juice-resistant polymeric layered material partially neutralized with alkali and having cation exchange capacity and a gastric juice-resistant outer layer.

Described herein are pharmaceutical compositions that comprise about 0.1 to about 1000 mg or up to 2000 mg or up to 3000 mg (or any value between 0.1-3000 mg), in the form of immediate release tablets for oral administration. The pharmaceutical compositions further comprise inactive ingredients such as flavoring agents, copovidone, ethylcellulose, magnesium stearate, mannitol, and silicondioxide.

Described herein are pharmaceutical compositions that comprise about 0.1 to about 1000 mg or up to 2000 mg or up to 3000 mg (or any value there between), about 500 to about 1000 mg, about 500 to about 2000 mg, about 500 mg, about 750 mg, about 1000 mg, about 1500 mg, about 2000 mg in the form of extended release tablets for oral administration. The pharmaceutical compositions further comprise inactive ingredients such as ethylcellulose, dibutyl sebacate, polyvinyl pyrroliodone, sodium stearyl fumarate, colloidal silicon dioxide, and polyvinyl alcohol.

The pharmaceutical compositions disclosed herein may be disclosed in unit-dosage forms or multiple-dosage forms. Unit-dosage forms, as used herein, refer to physically discrete units suitable for administration to human and animal subjects and packaged individually as is known in the art. Each unit-dose contains a predetermined quantity of the oligosaccharide sufficient to produce the desired therapeutic effect, in association with the required pharmaceutical carriers or excipients. Examples of unit-dosage forms include ampoules, syringes, and individually packaged to capsules. Unit-dosage forms may be administered in fractions or multiples thereof. A multiple-dosage form is a plurality of identical unit-dosage forms packaged in a single container to be administered in segregated unit-dosage form. Examples of multiple-dosage forms include vials, bottles of tablets or capsules, or bottles of pints or gallons.

The oligosaccharides as disclosed herein may be administered alone, or in combination with one or more other oligosaccharides disclosed herein, and/or one or more other active ingredients. The pharmaceutical compositions that comprise an oligosaccharide disclosed herein may be formulated in various dosage forms for oral, parenteral, and topical administration. The pharmaceutical compositions may also be formulated as a modified release dosage form, including delayed-, extended-, prolonged-, sustained-, pulsatile-, controlled-, accelerated- and fast-, targeted-, programmed-release, and gastric retention dosage forms.

These dosage forms can be prepared according to conventional methods and techniques known to those skilled in the art (see, Remington: The Science and Practice of Pharmacy, supra; Modified-Release Drug Delivery Technology, Rathbone et al., Eds., Drugs and the Pharmaceutical Science, Marcel Dekker, Inc.: New York, N.Y., 2002; Vol. 126).

The pharmaceutical compositions disclosed herein may be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

Once improvement of the patient's condition has occurred, a maintenance dose is administered if necessary. Subsequently, the dosage or the frequency of administration, or both, can be reduced, as a function of the symptoms, to a level at which the improved disease, disorder or condition is retained. Patients can, however, require intermittent treatment on a long-term basis upon any recurrence of symptoms.

The pharmaceutical compositions disclosed herein may be formulated in solid, semisolid, or liquid dosage forms for oral administration. As used herein, oral administration also includes buccal, lingual, and sublingual administration. Suitable oral dosage forms include, but are not limited to, tablets, capsules, pills, troches, lozenges, pastimes, cachets, pellets, medicated chewing gum, granules, bulk powders, effervescent or non-effervescent powders or granules, solutions, emulsions, suspensions, solutions, wafers, sprinkles, elixirs, and syrups. In addition to the oligosaccharides, the pharmaceutical compositions may contain one or more pharmaceutically acceptable carriers or excipients, including, but not limited to, binders, fillers, diluents, disintegrants, wetting agents, lubricants, glidants, coloring agents, dye-migration inhibitors, sweetening agents, and flavoring agents.

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, Panwar gum, ghatti gum, mucilage of isabgol husks, carboxymethyl cellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethyl cellulose (HEC), hydroxypropyl cellulose (HPC), hydroxypropyl methyl cellulose((HPMC); microcrystalline celluloses, such as AVICEL-PH-101, AVICEL-PH-103, AVICEL RC-581, AVICEL-PH-105(FMC Corp., Marcus Hook, Pa.); and mixtures thereof. Suitable fillers include, but are not limited to, talc, calcium carbonate, microcrystalline cellulose, powdered cellulose, dextrates, kaolin, mannitol, silicic acid, sorbitol, starch, pre-gelatinized starch, and mixtures thereof. The binder or filler may be present from about 50 to about 99% by weight in the pharmaceutical compositions disclosed herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof. The amount of disintegrant in the pharmaceutical compositions disclosed herein varies upon the type of formulation, and is readily discernible to those of ordinary skill in the art. The pharmaceutical compositions disclosed herein may contain from about 0.5 to about 15% or from about 1 to about 5% by weight of a disintegrant.

It should be understood that many carriers and excipients may serve several functions, even within the same formulation. The pharmaceutical compositions disclosed herein may be formulated as compressed tablets, tablet triturates, chewable lozenges, rapidly dissolving tablets, multiple compressed tablets, or enteric-coating tablets, sugar-coated, or film-coated tablets.

The tablet dosage forms may be prepared from the active ingredient in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The pharmaceutical compositions disclosed herein may be formulated as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms disclosed herein may be encapsulated in a capsule. Suitable liquid and semisolid dosage forms include solutions and suspensions in propylene carbonate, vegetable oils, or triglycerides. Capsules containing such solutions can be prepared as described in U.S. Pat. Nos. 4,328,245; 4,409,239; and 4,410,545. The capsules may also be coated as known by those of skill in the art in order to modify or sustain dissolution of the active ingredient.

The pharmaceutical compositions disclosed herein may be formulated in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquids or solvent, emulsifying agent, and preservative.

Suspensions may include a pharmaceutically acceptable suspending agent and preservative. Aqueous alcoholic solutions may include a pharmaceutically acceptable acetal, such as a di(lower alkyl) acetal of a lower alkyl aldehyde (the term “lower” means an alkyl having between 1 and 6 carbon atoms), e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing the active ingredient(s) disclosed herein, and a dialkylated mono- or poly-alkylene glycol.

The pharmaceutical compositions disclosed herein for oral administration may be also formulated in the forms of liposomes, micelles, microspheres, or nanosystems. Micellar dosage forms can be prepared as described in U.S. Pat. No. 6,350,458.

The pharmaceutical compositions disclosed herein may be formulated as non-effervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide.

The pharmaceutical compositions disclosed herein can be formulated as an oral nutritional composition. An oral nutritional composition can contain sources of protein, lipids and/or digestible carbohydrates and can be in solid, powdered or liquid forms. The composition can be designed to be the sole source of nutrition or a nutritional supplement. Suitable protein sources include intact, hydrolyzed, and partially hydrolyzed protein, which can be derived from any suitable source such as milk (e.g., casin, whey), animal (e.g., meat, fish), cereal (e.g., rice, corn), and vegetable (e.g., soy, potato, pea), insect (e.g., locust) and combinations of these sources.

Examples of the source of protein include whey protein concentrates, whey protein isolate, whey protein hydrolysates, and acid.

The pharmaceutical compositions disclosed herein may be formulated as immediate or modified release dosage forms, including delayed-, sustained, pulsed-, controlled, targeted-, and programmed-release forms.

The pharmaceutical compositions disclosed herein may be co-formulated with other active ingredients which do not impair the desired therapeutic action, or with substances that supplement the desired action.

The pharmaceutical compositions disclosed herein may be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, and subcutaneous administration.

The pharmaceutical compositions disclosed herein may be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, Remington: The Science and Practice of Pharmacy, supra).

The pharmaceutical compositions intended for parenteral administration may include one or more pharmaceutically acceptable carriers and excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

The pharmaceutical compositions disclosed herein may be formulated for single or multiple dosage administration. The single dosage formulations are packaged in an ampule, a vial, or a syringe. The multiple dosage parenteral formulations must contain an antimicrobial agent at bacteriostatic or fungistatic concentrations. All parenteral formulations must be sterile, as known and practiced in the art.

The pharmaceutical compositions may be formulated as a suspension, solid, semi-solid, or thixotropic liquid, for administration as an implanted depot. In one embodiment, the pharmaceutical compositions disclosed herein are dispersed in a solid inner matrix, which is surrounded by an outer polymeric membrane that is insoluble in body fluids but allows the active ingredient in the pharmaceutical compositions diffuse through.

Pharmaceutically acceptable carriers and excipients suitable for use in the topical formulations disclosed herein include, but are not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, penetration enhancers, cryoprotectants, lyoprotectants, thickening agents, and inert gases.

The pharmaceutical compositions disclosed herein may be administered intranasally or by inhalation to the respiratory tract. The pharmaceutical compositions may be formulated in the form of an aerosol or solution for delivery using a pressurized container, pump, spray, atomizer, such as an atomizer using electrohydrodynamics to produce a fine mist, or nebulizer, alone or in combination with a suitable propellant, such as 1,1,1,2-tetrafluoroethane or 1,1,1,2,3,3,3-heptafluoropropane. The pharmaceutical compositions may also be formulated as a dry powder for insufflation, alone or in combination with an inert carrier such as lactose or phospholipids; and nasal drops. For intranasal use, the powder may comprise a bioadhesive agent, including chitosan or cyclodextrin.

The pharmaceutical compositions disclosed herein may be formulated as a modified release dosage form. As used herein, the term “modified release” refers to a dosage form in which the rate or place of release of the active ingredient(s) is different from that of an immediate dosage form when administered by the same route.

Modified release dosage forms include delayed-, extended-, prolonged-, sustained-, pulsatile-, controlled-, accelerated- and fast-, targeted-, programmed-release, and gastric retention dosage forms. The pharmaceutical compositions in modified release dosage forms can be prepared using a variety of modified release devices and methods known to those skilled in the art, including, but not limited to, matrix controlled release devices, osmotic controlled release devices, multiparticulate controlled release devices, ion-exchange resins, enteric coatings, multilayered coatings, microspheres, liposomes, and combinations thereof. The release rate of the active ingredient(s) can also be modified by varying the particle sizes and polymorphism of the active ingredient(s).

The pharmaceutical compositions disclosed herein in a modified release dosage form may be prepared by methods known to those skilled in the art, including direct compression, dry or wet granulation followed by compression, melt-granulation followed by compression.

Generally, the amount of an oligosaccharide disclosed herein required to be administered to the person can vary depending upon factors such as the risk and condition severity, the age of the person, the form of the composition, and other medications being administered to the person. It would be expected that an oligosaccharide described herein should be well tolerated irrespective of the age and condition of the subject. The dosage of oligosaccharide to be administered can readily be set by a medical practitioner and would generally be in the range from about 100 mg to about 20 g per day, in certain embodiments from about 100 mg to about 15 g per day, from about 500 mg to about 15 g per day, in certain embodiments from about 500 mg to about 10 g per day, in certain embodiments from about 1 g to about 7.5 g per day. An appropriate dose can be determined based on several factors, including, for example, the body weight and/or condition of the patient being treated, the severity of the condition, being treated, other ailments and/or diseases of the person, the incidence and/or severity of side effects and the manner of administration. Appropriate dose ranges can be determined by methods known to those skilled in the art. During an initial treatment phase, the dosing can be higher (for example 200 mg to 20 g per day, preferably 500 mg to 15 g per day, more preferably 1 g to 10 g per day, in certain embodiments 2.5 g to 7.5 g per day). During a maintenance phase, the dosing can be reduced (for example, 10 mg to 10 g per day, preferably 100 mg to 7.5 g per day, more preferably 500 mg to 5 g per day, in certain embodiments 1 g to 2.5 g per day).

Depending on the disorder to be treated and the injection in suitable dosage unit with pharmaceutically acceptable carriers, adjuvants and vehicles appropriate for each route of administration.

The dose may be in the form of one, two, three, four, five, six, or more sub-doses that are administered at appropriate intervals per day. The dose or sub-doses can be administered in the form of dosage units containing from about 0.01 to about 2 grams, from about 0.05 to about 1 gram, or from about 10 to about 500 milligrams active ingredient(s) per dosage unit.

In certain embodiments, an appropriate dosage level is about 0.01 to about 5 g/kg patient body weight per day (mg/kg per day), about 0.01 to about 1 g/kg per day, about 0.01 to about 0.5 g/kg per day, or about 0.1 to about 500 mg/kg per day, which may be administered in single or multiple doses. A suitable dosage level may be about 0.1 to about 500 mg/kg per day, about 0.1 to about 250 mg/kg per day, or about 0.1 to about 100 mg/kg per day. Within this range the dosage may be about 0.01 to about 0.1, about 0.1 to about 1.0, about 1.0 to about 10, or about 10 to about 100 mg/kg per day.

The oligosaccharides disclosed herein may also be combined or used in combination with other agents useful in the treatment, prevention, or amelioration of one or more symptoms of a condition as described herein. Or, by way of example only, the therapeutic effectiveness of one of the oligosaccharides described herein may be enhanced by administration of an adjuvant (i.e., by itself the adjuvant may only have minimal therapeutic benefit, but in combination with another therapeutic agent, the overall therapeutic benefit to the patient is enhanced).

Such other agents, adjuvants, or drugs may be administered, by a route and in an amount commonly used therefore, simultaneously or sequentially with an oligosaccharide as disclosed herein. When an oligosaccharide as disclosed herein is used contemporaneously with one or more other drugs, a pharmaceutical composition containing such other drugs in addition to an oligosaccharide disclosed herein may be utilized but is not required.

Accordingly, the pharmaceutical compositions disclosed herein include those that also contain one or more other active ingredients or therapeutic agents, in addition to an oligosaccharide disclosed herein.

While this invention has been particularly shown and described with references to preferred embodiments thereof, it will be understood by those skilled in the art that various changes in form and details may be made therein without departing from the scope of the invention encompassed by the appended claims.

1. A method for the treatment of long COVID in a patient in need thereof, the method comprising administering to said patient a composition comprising a therapeutically effective amount of at least one human milk oligosaccharide, wherein such treatment reduces or improves at least one sign or symptom of long COVID.

2. The method of claim 1, wherein the at least one human milk oligosaccharide is selected from the group consisting of lacto-N-tetraose (LNT), lacto-N-neotetraose (LNnT), lacto-N-hexaose (LNH), lacto-N-neohexaose (LNnH), 2′fucosyllactose (2′FL), 3′fucosyllacose (3′FL), lacto-difucotetraose (LDFT), lacto-N-fucopentaose II/III (LNFP I/III), lactose-N-fucopentaose I (LNFP I), lacto-N-difuco-hexaose I (LNDFH I), lacto-N-difuco-hexaose II (LNDFH II), difucosyl-para-lacto-N-neohexaose (DFpLNnH), difucosyllacto-N-hexaose c (DFLNH c), 3′sialyllactose (3′SL), 6′sialyllactose (6′SL), LS-tetrasaccharide a (LSTa), LS-tetrasaccharide b (LST b), LS-tetrasaccharide c (LST c), 3′-sialyl-N-acetyllactosamine (3′SLN), 6′-sialyl-N-acetyllactosamine (6′SLN), or disialyllacto-N-tetraose (DSLNT).

3. The method of claim 2, wherein the one or more human milk oligosaccharide selected from 2′FL, 3′FL, 3′SL, 6′SL, LNT, or LNnT.

4. The method of claim 1, wherein the composition comprising one or more human milk oligosaccharides contains a mixture of two, three, four or five human milk oligosaccharides.

5. (canceled)

6. The method of claim 1, wherein the composition comprises a mixture of one neutral core and one neutral fucosylated human milk oligosaccharide, or comprises a mixture of one neutral and one acidic human milk oligosaccharide.

7. (canceled)

8. The method of claim 2, wherein the one or more human milk oligosaccharides is selected from 2′FL, 3′SL or 6′SL.

9. (canceled)

10. The method of claim 1, wherein the composition is not a mammalian milk and is not derived from mammalian milk.

11. (canceled)

12. The method of claim 1, wherein the composition comprises 10% or more, 20% or more, 30% or more, 40% or more, or 50% or more by mass one or more human milk oligosaccharides.

13. The method of claim 1, wherein the at least one symptom is selected from the group consisting of brain fog or other neurological signs and symptom; depression, anxiety, or other neuropsychiatric symptom; fatigue; muscle weakness; dyspnea, cough, or other respiratory symptom; insomnia or sleep disturbance; headache; paresthesias; dysautonomia; olfactory or gustatory dysfunction; hair loss or other dermatologic symptom; palpitations/tachycardia, chest pain, or other cardiac symptom; arthralgia/joint pain; rhinitis; Sicca/Sjogren syndrome; fever; and gastroenterological symptom; or a combination of any of thereof.

14. The method of claim 13, wherein the treatment reduces fatigue as measured by visual analogue scale, Samn-Perelli seven point fatigue scale, Karolinska Sleepiness Scale, or Psychomotor Vigilance Task; and/or reduces muscle weakness as measured by a test selected from the group consisting of lower handgrip muscle strength, short physical performance battery (SPPB) score, Timed Up and Go test (TUGT), walking speed (WS), and/or grip strength (GS).

15. The method of claim 13, wherein the treatment reduces brain fog as measured by the cognitive battery score.

16. The method of claim 13, wherein the treatment reduces a neurological sign or symptom, wherein the neurological sign or symptom is memory impairment as measured by a test selected from Short Test of Mental Status, the Montreal Cognitive Assessment (MoCA) or the Mini-Mental State Examination (MMSE), or by an assessment of symptoms by a physician.

17. The method of claim 13, wherein the treatment improves insomnia and/or sleep disturbance as measured by the Karolinska Sleep Scale (KSS) or the Sleepiness Symptoms Questionnaire (SSQ).

18. The method of claim 13, wherein the treatment reduces dyspnea as measured by a clinical scale selected from the group consisting of the Medical Research Council (MRC) scale, the Baseline Dyspnea Index (BDI), and Transitional Dyspnea Index (TDI), or a combination thereof.

19. The method of claim 13, wherein the treatment reduces depression and/or anxiety and/or other neuropsychiatric symptoms, reduces or improves headache, disautonomias, and/or paresthesias, or improves olfactory dysfunction or gustatory dysfunction.

20. (canceled)

21. (canceled)

22. The method of claim 13, wherein the method reduces hair loss or other dermatologic symptoms, palpitations, or joint pain/arthralgia.

23. (canceled)

24. (canceled)

25. (canceled)

26. The method of claim 1, wherein the method improves gut microbiome dysbiosis.

27. (canceled)

28. (canceled)

29. (canceled)

30. (canceled)

31. The method of claim 1, wherein said long COVID patient had mild, moderate or severe acute COVID infection during the acute phase of infection.

32. The method of claim 31, wherein the patient had mild or moderate COVID-19.

33. The method of claim 32, wherein the patient had COVID-19 not requiring hospitalization.

34. The method of claim 1, wherein the patient is 55 years old or less.

35. (canceled)

36. The method of claim 1, wherein the patient is 55 years or older.

37. (canceled)

38. (canceled)

39. The method of claim 1, wherein the patient is female.

40. The method of claim 1, wherein the patient is male.

41. The method of claim 1, wherein the patient has a body mass index (BMI) greater than or equal to 30.

42. The method of claim 1, wherein the patient experienced at least five symptoms during the first week of infection.

43. The method of claim 1, wherein said long COVID patient was prescribed antibiotics during the acute phase of infection.

44. The method of claim 1, wherein the patient has been vaccinated against SARS-CoV-2.

45. The method of claim 1, wherein the patient has not been vaccinated against SARS-CoV-2.

46. (canceled)

47. (canceled)

48. The method of claim 1, wherein the reduction and/or improvement of at least one sign or symptom of long COVID is assessed by a method comprising use of the Work Productivity and Activity Impairment (WPAI) Questionnaire.
